(19) United States
(12) Patent Application Publication
(10) Pub. No.: US 2008/0166741 A1
(43) Pub. Date: Jul. 10, 2008

(54) Method for Diagnosing Membranous Glomerulonephritis Disease and a Kit Thereof

(75) Inventors: Tzu-Ling Tseng, Chiayi City (TW); Pei-Hsiu Liao, Taipei City (TW); Ping-Fu Cheng, Sijhou Township, Changhua County (TW); Chi-Hung Cheng, Taichung City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Industrial Technology Research Institute, Chutung Town (TW)

(21) Appl. No.: 11/620,064

(22) Filed: Jan. 5, 2007

(51) Int. Cl. G01N 33/53; G01N 33/00

(52) U.S. Cl. 435/7.92; 435/7.1

(57) ABSTRACT

Some specific autoantigens of MGN are discovered by using the integrated platform. Moreover, according to the specificity between antibodies and antigens, a method for diagnosing MGN are constructed. This diagnostic method is cooperated with ELISA, RIA, immunofluorescence, or other immunochromatography techniques. Finally, a diagnostic kit is provided to implement the method above.

### BACKGROUND

1. Field of Invention

The present invention relates to diagnose an autoimmune disease. More particularly, the present invention relates to a method for diagnosing membranous glomerulonephritis disease and a kit thereof.

2. Description of Related Art

Autoimmune diseases result from aberrant immune cell function or activity which causes inappropriately activated T cells to react against self tissue, thereby triggering production of cytokines and/or autoantibodies responsible for disease etiology and progression (Cotran et al., Pathologic Basis of Disease 211-212 (6th ed. 1999); Scofield, “Autoantibodies as predictors of disease”, Lancet 363:1544-1546 (2004)). Autoimmune disorders may be systemic which affects multiple organs or tissues, or localized that only affects a single organ, organ system or tissue.

A hallmark of autoimmune disease is the production of high affinity autoantibodies directed against self proteins (Robinson et al., “Autoantigen microarrays for multiplex characterization of autoantibody responses,” Nature Med. 8(3):295-301 (2002)). This is also called “Autoimmunity”, a loss of self-tolerance. As a result of serum autoantibodies often appearing long before the onset of clinical symptoms (Scofield “auatoantibodies as predictor of disease”, Lancet 2004; 363:1544-46), the specificity and pathogenicity of autoantibodies in respons to a certain disease highlight their potential as important tools for improved diagnosis, classification and prognostication.

Membranous glomerulonephritis (MGN) is one of the autoimmune disease, which is also called membranous nephropathy. MGN is a renal disorder which causes the disruption of kidney function due to the inflammation of the glomerulus, and the changes in the glomerular basement membrane. The disorder occurs in approximately 2 out of 10,000 people. It may occur at any age but is more common after age 40 (Medline Plus Medical Encyclopedia: Membranous nephropathy, last accessed Sep. 13, 2005, updated by Robert Mushnick, M.D.). Several symptoms have been observed associated with MGN, such as reveal protein in the urine, blood in the urine (hematuria), or increasing blood lipid from urinalysis and serum analysis. Besides, the presence of laminin β-1 chain within the glomerular basement membrane (GMB) of MGN patients could play a role in the occurrence of proteinuria (Fischer E et al., “Abnormal expression of glomerular basement membrane laminins in membranous glomerulonephritis”, Nephrol Dial Transplant 15(12): 1956-6). In addition, the repeated administration of mercury to Brown Norway (BN) rats induces the production of autoantibodies to laminin 1 and other autoantigens, accompanied by renal deposition of immunoglobulins and a membranous glomerulonephropathy (Bigazzi PE et al. Clin Immunol. 109(2): 229-37). However, the detail mechanism of MGN is still unknown.

At present, the diagnosis of MGN is still confirmed by renal biopsy. Renal biopsy is an invasive medical treatment, wherein pathology of IgG deposition from glomerular subepithelia to intramembrane is observed in MGN patient's specimen. However, doing biopsy has higher risk and may cause complications. Therefore, it is necessary to develop other methods for diagnosing MGN.

### SUMMARY

A method for diagnosing MGN is provided. First, a biological sample is obtained from a subject. Then, the presence of at least one autoantibody in the biological sample is detected, wherein the autoantibody specifically binds at least one protein selected from laminin alpha-1 chain precursor, glycogen phosphorylase, catalase, α-amylase, and glyceraldehyde-3-phosphate dehydrogenase. The presence of the autoantibody in the biological sample indicates that the subject has membranous glomerulonephritis disease.

A kit for identifying a subject having membranous glomerulonephritis disease is further provided. The kit comprise reagents for determining presence of at least one autoantibody that specifically binds one or more proteins selected from laminin alpha-1 chain precursor, glycogen phosphorylase, catalase, α-amylase, and glyceraldehyde-3-phosphate dehydrogenase in a biological sample.

Accordingly, by detecting the presence of autoantibodies that specifically bind to the autoantigens characteristic of MGN disease, MGN disease can be detected. Several methods are used for detecting the presence of autoantibodies such as Western blot, enzyme-linked immunosorbent assay (ELISA), or radiation immune assay (RIA). Furthermore, the method provided above only requires biological samples, such as serum, blood, plasma, saliva, amniotic fluid, synovial fluid, lacrimal fluid, milk, lymph, urine, sweat, and combination thereof.

It is to be understood that both the foregoing general description and the following detailed description are by examples, and are intended to provide further explanation of the invention as claimed.

### BRIEF DESCRIPTION OF THE DRAWINGS

These and other features, aspects, and advantages of the present invention will become better understood with regard to the following description, appended claims, and accompanying drawings where:

FIG. 1 depicts the flow chart of the experiment of the embodiment according to an embodiment of the present invention;

FIGS. 2A-2C depicts the flow chart for the autoantigens screening method according to an embodiment of the present invention;

FIGS. 2D-2F depicts the flow chart for the MGN specific autoantigen screening process of one embodiment of the present invention;

FIG. 3 depicts the flow chart of the validation of Western blot according to an embodiment of the present invention.

FIG. 4 depicts the validation result of catalase by Western blot according to an embodiment of the present invention;

FIG. 5 depicts the validation result of GAPDH by Western blot according to an embodiment of the present invention;

FIG. 6 depicts the validation result of α-amylase by Western blot according to an embodiment of the present invention; and

FIG. 7 depicts the validation result of glycogen phosphorylase by Western blot according to an embodiment of the present invention.

### DETAILED DESCRIPTION

Reference will now be made in detail to the present embodiments of the invention, examples of which are illustrated in the accompanying drawings.

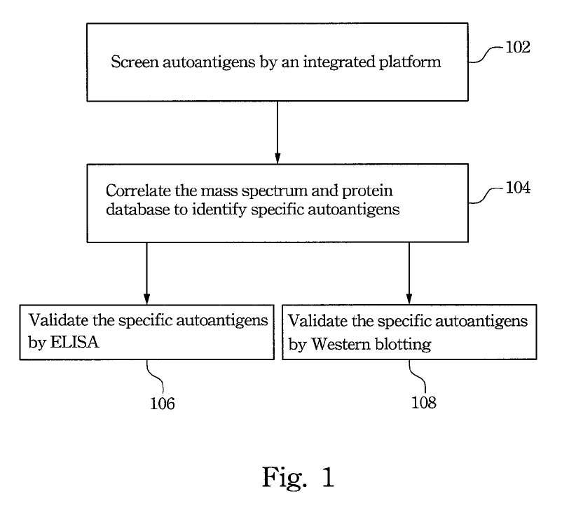

According to in FIG. 1, it depicts the general procedures according to an embodiment of the present invention. First, an integrated platform is used to screen autoantigens (step 102) whereby non-specific autoantigens are removed and only those specific autoantigens remain. The integrated platform herein was established in US 2005/0124076 A1, “Method for screening autoantigen”.

Then, by correlating the mass spectrum and NCBlnr protein database, the specific autoantigens are identified (step 104). Finally, the specific autoantigens, which relate to MGN, are further validated by ELISA (step 106) and/or Western blot (step 108), repsectively. All detail procedures are presented as follows.

(A) Sample Preparation

Autoantigens for screening were obtained from human plasma and renal adenocarcinoma cell extract. All samples may be analyzed immediately after being extracted or after stored, wherein “stored” means that the samples may be equilibrated with an appropriate storage buffer, and kept below or equivalent to 4° C., such as 4° C., −20° C., −70° C., or even in cryogenic liquid, such as liquid nitrogen.

In the embodiment of the present invention, renal adenocarcinoma cell 786-O (BCRC No. 60243, a common renal cancer cell line) was purchased from Food Industry Research And Development Institute, Taiwan, Republic of China. The lysate of cell 786-O was extracted as following steps: first, the cells were incubated with lysis buffer (15 mM Tris-HCl, 120 mM NaCl, 25 mM KCl, 2 mM EDTA, 0.5% Triton X-100, 0.1 M DTT, 0.5 mM PMSF, 10 μg/mL leupeptin, phosphatase inhibitor I & II, pH 7.5) at 4° C. for 30 minute (min). Next, the sample was centrifuged with 14000 rpm at 4° C. for 15 min. Finally, the supernatant was collected for screening.

Moreover, autoantibodies used to screen autoantigens may be obtained from serum, blood, plasma, saliva, amniotic fluid, synovial fluid, lacrimal fluid, milk, lymph, urine, sweat and combination thereof. In an embodiment of the present invention, autoantibodies were from serum samples.

Serum samples from healthy individuals and patients who suffering from the unrelated immune disorder or myasthenia gravis (MG) were used as negative controls. The healthy individuals were from Show Chwan Health Care System or Taichung Veterans General Hospital, Taiwan, Republic of China, and the patients were from Shin-Kong Hospital, Taiwan, Republic of China.

MGN Serum samples were obtained from 77 membranous glomerulonephritis patients at Taichung Veterans General Hospital, Taiwan, Republic of China, wherein 14 serum samples were used as screening autoantibodies, and 63 serum samples are used for ELISA and Western blot.

(B) Screening Autoantigens by an Integrated Platform

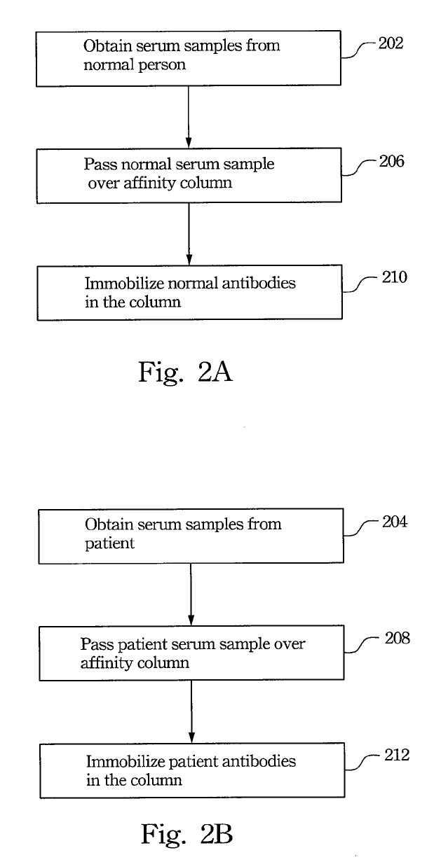

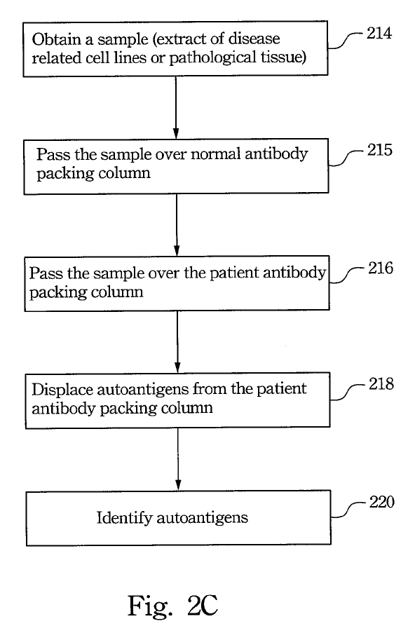

According to US 2005/0124076 A1, “Method for screening autoantigen”, which is incorporated here entirely by reference, the integrated platform was established to provide a method for screening autoantigens. The following FIGS. 2A-2C illustrate the principle of the integrated platform. As shown in FIG. 2A, serum samples are obtained from normal person first (step 202). Then, the samples are passed over an affinity column that can capture autoantibodies (e.g. Protein G affinity column or Protein A affinity column) in order to purify the autoantibodies contained in the serum samples (step 206). After that, the purified autoantibodies are immobilized in a column (step 210) to obtain normal antibody packing column.

FIG. 2B illustrates the procedures to obtain a patient antibody packing column. Similarly, serum samples are obtained from patients first (step 204). Next, the samples are passed over an affinity column that capture autoantibodies (e.g. Protein G affinity column or Protein A affinity column) in order to purify the autoantibodies contained in the serum samples (step 208). Finally, the purified autoantibodies are immobilized in a column (step 212) to obtain patient antibody packing column.

The general principle of screening autoantigens by using both normal and patient antibody packing columns are illustrated in FIG. 2C. First, a sample, such as an extract of disease related cell lines or pathological tissue, is obtained (step 214). Second, the sample is passed over the normal antibody packing column (step 215) to remove the non-specific autoantigens from the sample. Then, the sample is passed over the patient antibody packing column to immobilize some specific autoantigens (step 216). After that, these autoantigens retained in patient antibody packing column are displaced (step 218), and finally the specific autoantigens obtained from the patient antibody packing column are identified (step 220).

According to the principle shown in FIGS. 2A-2C, FIGS. 2D-2F illustrate one embodiment of the present invention which used the integrated platform to screen MGN specific autoantigens. All operating steps of columns (Protein G column, normal human serum-activated (NHS-activated) column, and antibody packing column) followed the protocols provided by GE Healthcare. Referring to FIG. 2D, 7 serum samples were obtained from normal people first (step 222). Then, the samples were passed over a protein G column (HiTrap Protein G HP, P/N 17-0404-01, GE Healthcare) (step 224) to purify the IgG contained in the sera.

Finally, the purified IgG were coupled in a NHS-activated column (HiTrap NHS-activated HP, P/N 17-0716-01, GE Healthcare) (step 226) for immobilized. At this step, a normal antibody packing column was obtained, and used as an affinity column for capturing autoantigens not specific to MGN.

FIG. 2E illustrates the procedures to obtain a patient antibody packing column. Similarly, 14 serum samples were prepared from MGN patients first (step 228). Next, the samples were passed over a protein G column (HiTrap Protein G HP, P/N 17-0404-01, GE Healthcare) to purify IgG from the sera (step 230). Finally, the purified IgG were coupled in a NHS-activated column (HiTrap NHS-activated HP, P/N 17-0716-01, GE Healthcare) (step 232) to construct a patient antibody packing column used as an affinity column for specific MGN autoantigens.

According to FIG. 2F, it illustrates the procedures of screening autoantigens by both normal and patient antibody packing columns. First, the lysate of cell 786-O was extracted as described above (step 234). Then, the lysate was loaded into the normal antibody packing column (step 236) and over the patient antibody packing column (step 238) successively. Thus, the non-specific autoantigens in the sample were kept in the normal antibody packing column, and the specific autoantigens related to MGN were captured in the patient antibody packing column. After that, the specific autoantigens retained in patient antibody packing column were eluted (step 240), and then loaded into the normal antibody packing column again (step 242) to remove non-specific autoantigens completely. Finally, these purified specific autoantigens were identified by Mass spectrometry (step 244) in the following section (C).

(C) Mass Spectrometry Analysis

After obtaining the specific autoantigens, they were identified by mass spectrometry. First, these specific autoantigens were hydrolyzed by trypsin (P/N V5111, Promega) overnight. The hydrolyzed sample was initially separated by a HPLC system and analyzed by a MS/MS spectrometer to determine the amino acid sequences of the autoantigens. The HPLC was purchased from Dionex Corporation, and the column was of C18, P/N 160321, LC Packings. The MS/MS, which was a quadrupole-collision cell time-of-flight mass spectrometer, named Q-star, using electron-spray-ionization (ESI) was purchased from Applied Biosystems.

Finally, by correlating the MS/MS spectra with the information contained in the NCBlnr protein database using the Matrix Science Mascot ( v1.8 software, protein identification was achieved, wherein human specificity was also provided. The identification result of these specific autoantigens is listed in Table 1 as follows, wherein the Mascot score and the peptides matched are also provided. Mascot is a protein identification program by searching sequence databases using mass spectrometry data. The fundamental approach is to calculate the probability that the observed match between the experimental data set and each sequence database entry is a chance event. The match with the lowest probability (P) is reported as the best match. Since the probability for a good match is usually a small number, it must be expressed in scientific notation. Accordingly, the Mascot score is calculated as-10*Log (P), where P is the probability of a random observed match. This means that the best match is the one with the highest Mascot score. (David N. Perkins et al., “Probability-based protein identification by searching sequence databases using mass spectrometry data”, Electrophoresis 1999, 20, 3551-3567) Mascot scores>33 indicate identity or extensive homology (P<0.05). In addition, the peptides matched indicate the match extent between the MS/MS experimental data and the amount of peptides in the database. The higher amount matched, is the higher credibility.

According to Table 1, these autoantigenes include laminin alpha-1 chain precursor (337.1 KD), glycogen phosphorylase (96.9 kD), catalase (59.6 kD), α-amylase (57.7 kD), and glyceraldehyde-3-phosphate dehydrogenase (GAPDH, 35.9 kD).

TABLE 1
the result of autoantigens identification

| | | Peptides | Mascot |
|---|---|---|---|
| Name | MW (Da) | matched | score |
| Laminin alpha-1 chain precursor (Laminin | 337,153 | 4 | 34 |
| A chain) | | | |
| glycogen phosphorylase | 96,960 | 12 | 443 |
| Catalase | 59,625 | 2 | 52 |
| Human Pancreatic α-Amylase | 57,707 | 7 | 405 |
| glyceraldehyde-3-phosphate | 35,922 | 3 | 76 |
| dehydrogenase | | | |

(D) Validation of by Western Blot

In order to confirm the screening result above, four of the autoantigens, GAPDH, catalase, α-amylase, and glycogen phosphorylase, were confirmed by ELISA first. According to the validation result of ELISA, it showed that the absorbance of MGN specimen was much higher than the absorbance of control. This indicates that there were more antigen-antibody complexes formed in the MGN specimens due to loss of self-tolerance. Hence, these specific autoantigens screened by the integrated platform such as GAPDH, catalase, α-amylase, and glycogen phosphorylase are characteristic of MGN disease.

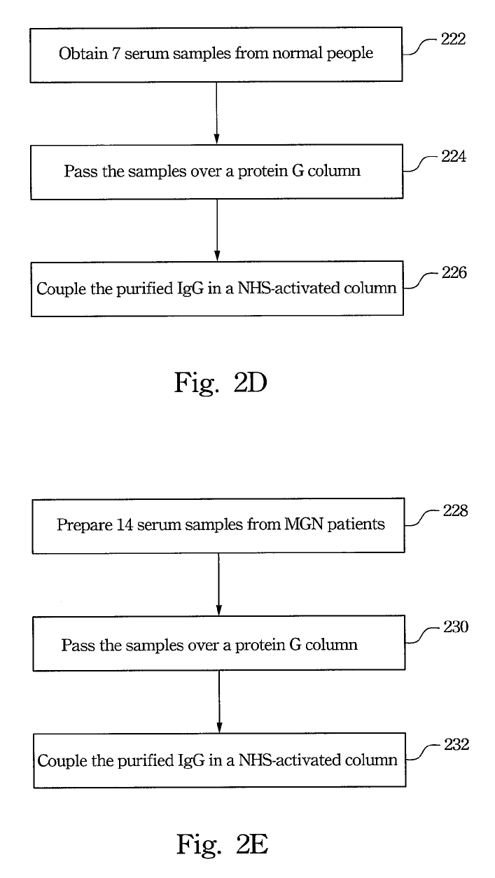

Next, the ELISA validation result was further verified by Western blot as illustrated in FIG. 3. First, 20 μg commercial proteins were separated on one piece of 10% SDS-PAGE gel by electrophoresis at 80 V in the stacking gel and 120 V in the resolving gel (step 302). These commercial proteins were glycogen phosphorylase (rabbit, Sigma P6635), catalase (Human sapiens, Sigma C3556), α-amylase (Human sapiens, Sigma A9972), and GAPDH (Human sapiens, Sigma G6019). Then, the fractionated proteins were transferred to polyvinylidene difluoride (PVDF) by TE70 Series Semi-dry Transfer unit (Amershan Bioscience) according to the manufacturer's instructions (step 304). The PVDF membrane has high binding affinity for proteins; therefore, it captures the autoantigens transferred from the gel. The transfer buffer here was Towbin buffer (1X is 25 mM Tris-HCl, pH 8.30, 192 mM glycine, and 20% (v/v) methanol).

Next, the PVDF membranes was soaked in the second blocking solution, 5% (w/v) powdered non-fat milk in PBST buffer (8 mM Na₂HPO₄, 2 mM NaH₂PO₄, 150 mM NaCl, 0.05% (v/v) Tween-20, pH 7.4), for 1 hr at room temperature (step 306) to block non-specific protein binding sites on the PVDF membrane. After that, the blocked membrane was incubated in 5% (w/v) powdered non-fat milk in PBST buffer which contains a 1:1000 dilution of patient's sera, for 1 hr at room temperature (step 308). Again, since the autoantibodies in MGN patient's sera lose self-tolerance, the autoantibodies will bind to autoantigens that are specific to MGN.

After incubated with primary antibodies (autoantibodies in patients' sera) the membrane was washed with PBST 3 times for 10 mins each (step 310) to remove unbound primary antibodies. Then the membrane was incubated with the secondary antibody, horseradish peroxidase-conjugated mouse anti-human IgG specific for F_{CY} (Jackson ImmunoResearch Laboratories, Inc.) 1 hr at room temperature (step 312). As mention above, this secondary antibody specifically bound to the primary antibody and functions as a detector.

Finally, the membrane was washed again with PBST 3 times for 10 mins each (step 314). Then the autoantibodies that bound to immobilized autoantigens were detected by enzyme-linked chemiluminescence using the ECL blotting substrate (Amersham Pharmacia Biotech), according to the manufacture's instructions (step 316).

(E) Validation Result of Western Blot

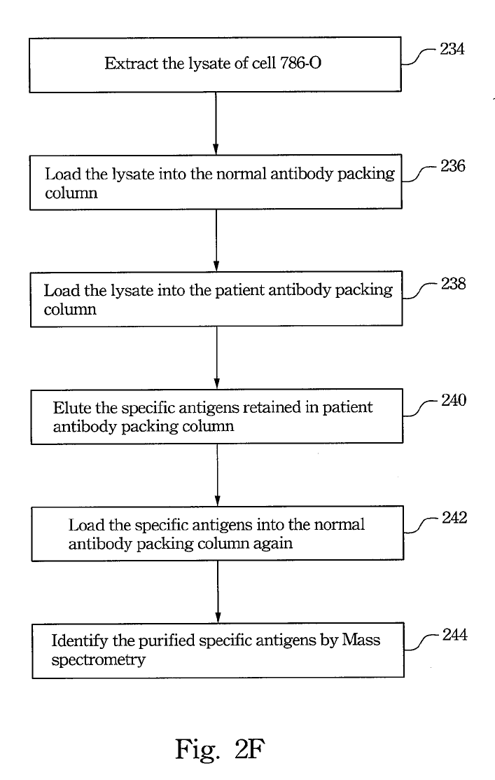

FIGS. 4-7 shows the validation results of Western blot. First, FIG. 4 illustrated the result of catalase. There were 10 sera samples used as controls (6 sera samples from health individuals and 4 sera samples from MG patients) and only two of them are shown in FIG. 4 as indicated. Since there was no band displayed on the PVDF membrane, it indicates that no antigen-antibody complex forms on the PVDF membrane. Thus, the autoantibodies in the control sera samples were not specific to catalase. In addition, there were 15 MGN patient's sera were also used as the primary antibody in Western blot, and only 4 of them were shown in FIG. 4 as indicated. According to FIG. 4, the MGN specimens display bands on the PVDF membrane. This indicates that antigen-antibody complexes formed on the PVDF membrane. The result shows that the autoantibodies in MGN patient's sera are much more specific to catalase as a result of loss of self-tolerance.

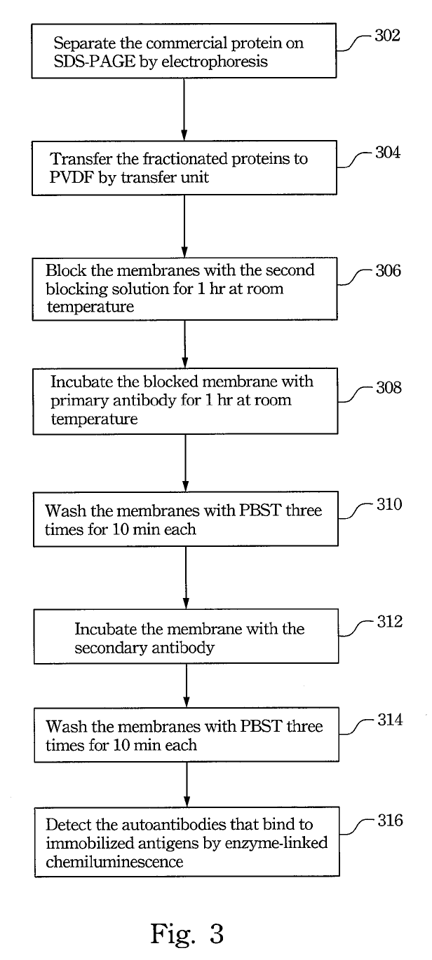

Next, FIG. 5 also exhibited the similar results for GAPDH. There were 28 control samples (13 from health individuals and 15 from MG patients), and only two of them were displayed in FIG. 5. Not surprisingly, no band displayed. Moreover, there were 25 MGN sera specimens used as the primary antibody, and 7 of them are shown in FIG. 5 as indicated. Again, the MGN specimens displayed bands on the PVDF membrane.

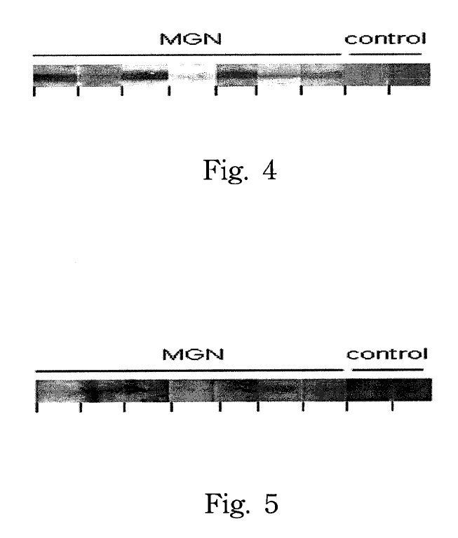

FIG. 6 exhibited the validation results of α-amylase. There were 24 controls, wherein 8 samples were from health individuals and 16 were from MG patients. Again, no bands revealed in FIG. 6 as indicated. In contrast, while MGN patient's sera were used as the primary antibody, bands are displayed on the PVDF membrane. Only 4 MGN specimen results are shownin FIG. 6.

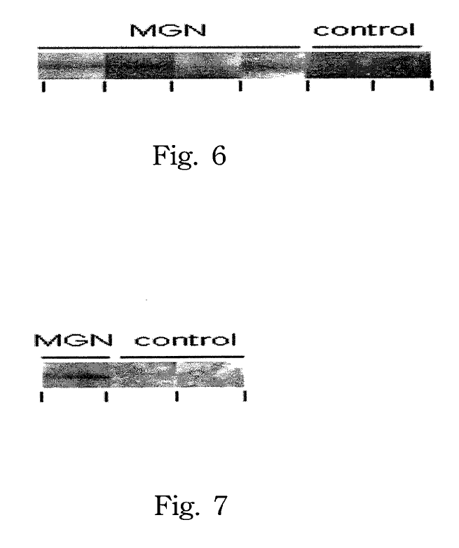

Finally, FIG. 7 exhibited the results for glycogen phosphorylase. There were 25 control samples (11 from health individuals and 14 from MG patients), and 25 MGN specimens used as the primary antibody. Again, MGN specimens displayed bands on the PVDF membrane.

Accordingly, the validation result of Western blot implies that the autoantibodies in MGN patient's sera are specific to catalase, GAPDH, α-amylase, and glycogen phosphorylase due to autoimmunity. Therefore, these autoantigens are characteristic of MGN disease. This not only confirms the screen result of the embodiment of the present invention, but also discloses that MGN disease can be diagnosed by detecting the specific autoantibodies in a biological sample, such as serum, blood, plasma, saliva, amniotic fluid, synovial fluid, lacrimal fluid, milk, lymph, urine, sweat, and combinations thereof.

Although the embodiments of the present invention above have been described in considerable detail with reference, other embodiments are possible. Therefore, their spirit and scope of the appended claims should no be limited to the description of the embodiments container herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method of diagnosing membranous glomerulonephritis disease in a subject comprising:
obtaining a biological sample from the subject; and
detecting at least one autoantibody in the biological sample, wherein the autoantibody specifically binds at least one protein selected from a group consisting of laminin alpha-1 chain precursor, glycogen phosphorylase, catalase, α-amylase, and glyceraldehyde-3-phosphate dehydrogenase,
when the autoantibody presents in the biological sample indicates that the subject has membranous glomerulonephritis disease.

2. The method of claim 1, wherein the biological sample is selected from a group consisting of serum, blood, plasma, saliva, amniotic fluid, synovial fluid, lacrimal fluid, milk, lymph, urine, sweat and combinations thereof.

3. The method of claim 1, wherein the autoantibody is detected by Western blot, enzyme-linked immunosorbent assay (ELISA), radiation immune assay (RIA) (lack support in the specification), or combinations thereof.

4. The method of claim 1, wherein the subject is a mammal.

5. The method of claim 4, wherein the mammal is a human.

6. A kit for identifying a subject having membranous glomerulonephritis disease comprising reagents for determining presence of at least one autoantibody that specifically binds one or more proteins selected from a group consisting of laminin alpha-1 chain precursor, glycogen phosphorylase, catalase, α-amylase, and glyceraldehyde-3-phosphate dehydrogenase in a biological sample.

7. The kit of claim 6, wherein the biological sample is selected from a group consisting of serum, blood, plasma, saliva, amniotic fluid, synovial fluid, lacrimal fluid, milk, lymph, urine, sweat and combinations thereof.

8. The kit of claim 6, wherein the autoantibody is detected by Western blotting, enzyme-linked immunosorbent assay (ELISA), radiation immune assay (RIA), or combinations thereof.

9. The kit of claim 6, wherein the subject is a mammal.

10. The kit of claim 9, wherein the mammal is a human.
